(19) United States
(12) Patent Application Publication
(10) Pub. No.: US 2006/0263319 A1
(43) Pub. Date: Nov. 23, 2006

(54) HAIR TREATMENT COMPOSITIONS

(76) Inventors: Shimei Fan, Inverness, IL (US); Natalia Nekludoff, Lisle, IL (US); Collen Katherine Costello, Chicago, IL (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 11/278,996

(22) Filed: Apr. 7, 2006

Related U.S. Application Data
(60) Provisional application No. 60/669,053, filed on Apr. 7, 2005.

(51) Int. Cl. A61K 8/73; A61K 8/41

(52) U.S. Cl. 424/70.13; 424/70.17; 424/70.28

(57) ABSTRACT

Disclosed are hair treatment compositions comprising: a) from 0.01 to 5 weight percent of cationic conditioning polymer; and b) from 0.5 to 5 weight percent of dicarboxylic acid of the formula:
HOOC-A-COOH
wherein A is an alkylene group of the formula: —(CH₂)ₙ— where n is an integer from 2 to 8, the alkylene group A being optionally substituted with a group of the formula —SO₃H, and, wherein from 0 to 100% of the dicarboxylic acid is a water-soluble salt thereof. Methods and system for treating hair using such compositions are also disclosed.

This application claims the benefit of U.S. provisional application No. 60/669,053 filed Apr. 7, 2005.

### FIELD OF THE INVENTION

This invention relates to hair treatment compositions. More particularly, the invention relates to hair treatment compositions comprising specific combinations of active materials. The compositions are particularly suitable for imparting volumizing and conditioning properties to hair.

### BACKGROUND OF THE INVENTION

A variety of agents are known to impart good conditioning properties to hair. However, conditioning is oftentimes imparted at the expense of body or volume, particularly in the case of fine, thin hair. One class of cationic conditioning polymers that has been found to be desirable in hair care products for body/volume seekers is quaternary cellulosics. Quaternary cellulosics include salts of hydroxyethyl cellulose reacted with trimethyl ammonium substituted epoxide, such as, for example, quaternary polymers referred to in the industry as Polyquaternium-10.

The inclusion of acids, including, for example, carboxylic acids, as pH adjusters in hair care compositions is known. As pH adjusters such acids are incorporated into hair care compositions at what are typically relatively low levels, for example, less than 0.5 wt. %, more commonly, from 0.01 to 0.3 wt. %.

The use of higher levels of carboxylic acids in hair care compositions has also been disclosed. U.S. Pat. No. 6,183,757 discloses what are therein characterized as mild, rinse-off antimicrobial cleansing compositions effective against Gram positive bacteria, gram negative bacteria, fungi, yeasts, molds and viruses, which compositions comprise:

(a) from about 0.001% to about 5% of an antimicrobial active,

(b) from about 1% to about 80% of an anionic surfactant;

(c) from about 0.1% to about 12% of a proton donating agent; and

(d) from about 3 to about 98.899% of water;

wherein the rinse-off antimicrobial cleansing compositions have a “One-wash Immediate Germ Reduction index” of greater than about 2.5, and a “Mildness Index” of greater than 0.3; the terms “One-wash Germ Reduction Index” and “Mildness Index” being as therein more particularly described. The patent notes that the proton donating agents can be organic acids, including polymeric acids, mineral acids or mixtures thereof. Listed as examples of organic acids are the following: adipic acid, tartaric acid, citric acid, maleic acid, malic acid, succinic acid, glycolic acid, glutaric acid, benzoic acid, malonic acid, salicylic acid, gluconic acid, polyacrylic acid, their salts, and mixtures thereof. The patent specifically exemplifies a hair shampoo composition comprising ammonium lauryl sulfate (7.00 wt. %) ammonium lareth-3 sulfate (9.00 wt. %), sodium lauroamphoacetate (5.00 wt. %), malic acid (2 wt. %), salicylic acid (2.00 wt %), sodium hydroxide (to pH 5.0), pyrithione zinc (1.00 wt. %), Polyquaternium 10 (0.50 wt. %) perfume (1.00 wt. %), dye (0.001%) and water (Q.S.).

WO93/25650 discloses highly concentrated (30-90%) surfactant concentrates that include an alkyl polyglycoside and an effective amount, e.g., about 0.1 to about 10% by weight, of a viscosity-adjusting agent selected from the group consisting of inorganic and organic electrolytes. Carboxylic acids and their salts are mentioned as organic electrolytes. The surfactant concentrates are disclosed as having utility for formulation into personal skin care products such as shampoos, foam baths, hand soaps, hair conditioners, facial cleansers and the like.

The use of sulfosuccinic acid in hair treatment compositions has been disclosed in publications that include copending U.S. patent application Ser. Nos. 10/894,891; 10/894,796; and 10/895,185, all filed Jul. 20, 2004. U.S. patent application Ser. No. 10/894,891, discloses a mild aqueous composition which is highly suitable for cleansing hair, which composition comprises:

i) from about 1% to about 20% of a sulfosuccinate surfactant,

ii) from about 1% to about 20% of an amphoteric surfactant, and

iii) a sulfosuccinic acid or a salt of a sulfosuccinic acid,

wherein the sulfosuccinic acid or the salt of sulfosuccinic acid is present in an amount of at least about 4% based on the weight of the sulfosuccinate surfactant. The level of sulfosuccinate surfactant present in the compositions is disclosed as being in the range from about 1% to about 20% by weight of the composition, preferably about 1% to about 10%, and most preferably from about 1.5% to about 7% of the composition. The application further discloses that the compositions may further comprise additives such as cationic polymers, including, for example, cationic cellulosic polymers.

U.S. patent application Ser. No. 10/894,796 discloses a mild aqueous composition that comprises:
i) a Mid-Chain alkyl ethoxy sulfosuccinate having an average alkyl chainlength between about 10 and about 14 carbon atoms and an average degree of ethoxylation between about 1 and about 5,
ii) an amphoteric surfactant,
iii) a Long-Chain alkyl ethoxy sulfosuccinate having an average alkyl chainlength of between about 16 and about 18 carbon atoms,
   wherein the Long-Chain alkyl ethoxy sulfosuccinate component iii) is present in the composition at a level from about 0.1% to about 6% based on the total weight of the Mid-Chain alkyl ethoxy sulfosuccinate component i). included among the Examples is provided in this application are compositions that comprise, among other components, lauryl ethoxy sulfate (1 EO), disodium laureth sulfosuccinate, disodium palmitoyl ethoxy sulfosuccinate (as a percentage of the dilaureth sulfosuccinate), cocamidopropyl betaine, and cationic guar, wherein sulfosuccinic acid is present as part of the added disodium laureth sulfosuccinate. The application further discloses that the compositions may further comprise additives such as cationic polymers, including, for example, cationic cellulosic polymers.

U.S. patent application Ser. No. 10/895,185, filed Jul. 20, 2004 discloses a mild aqueous composition which is highly suitable for cleansing hair and skin, which composition comprises:
i) from about 1% to about 20% of a sulfosuccinate surfactant;
ii) from about 1% to about 20% of an amphoteric surfactant; and
iii) an electrolyte at a level sufficient to provide at least about 0.1 equivalents of cationic ions per Kg of composition
   wherein the ratio of the sulfosuccinate surfactant to the amphoteric surfactant is in the range from about 2:1 to 1:2, and wherein the said electrolyte maintains a viscosity of the composition at its initial value after storage for 4 weeks at a temperature of 49° C. The application further discloses that the compositions may further comprise additives such as cationic polymers, including, for example, cationic cellulosic polymers.

It is an object of the present invention to provide a hair treatment composition that imparts a combination of good conditioning, and volume/body properties to hair, particularly fine, thin hair.

In at least one embodiment, it is a further object of this invention to provide a hair treatment composition that imparts body and fullness to the hair, particularly fine, thin hair, while leaving the hair feeling clean and non-greasy.

### SUMMARY OF THE INVENTION

It has now been found that the inclusion of both cationic conditioning polymer and relatively high levels of a C4 to C10 dicarboxylic acid and/or salt thereof, preferably a sulfonated dicarboxylic acid and/or salt thereof, provides hair treatment compositions having desirable conditioning and volume/body properties. Accordingly, in one embodiment of this invention there is provided a hair treatment composition comprising:
a) from 0.01 to 5 weight % of at least one cationic conditioning polymer; and
b) from 0.5 to 5 weight % of dicarboxylic acid of the formula:
   HOOC-A-COOH
   wherein A is an alkylene group of the formula: —(CH₂)ₙ—, where n is an integer from 2 to 8, the alkylene group A being optionally substituted with a group of the formula —SO₃H, and wherein from 0 to 100% of the dicarboxylic acid is a water-soluble salt thereof.

The compositions of this invention have particular application to the treatment and conditioning of fine, thin hair, but may also be used to add volume and body to all types of hair.

In a further embodiment, the present invention relates to a method of treating hair using the hair treatment compositions of this invention.

### DETAILED DESCRIPTION OF THE INVENTION

As used herein % or weight % refers to the percent by weight of an ingredient as compared to the total weight of the composition or component that is being discussed. Except in the operating and comparative examples, or where otherwise explicitly indicated, all numbers in this description indicating amounts of materials or conditions of reaction, physical properties of materials and/or use are to be understood as modified by the word “about”. All amounts are by weight of the final composition, unless otherwise specified. It should be noted that in specifying any range of concentration, any particular upper concentration can be associated with any particular lower concentration.

The final product form of hair treatment compositions according to the invention may suitably be, for example, shampoos, conditioners (particularly leave-on conditioners) and styling aids, with shampoo compositions being of particular interest.

The cationic conditioning polymer used in the practice of this invention may be a homopolymer or a copolymer formed from two or more types of monomers. The molecular weight of the polymer will generally be between 5,000 and 10,000,000, typically at least 10,000 and preferably in the range 100,000 to about 2,000,000. The polymers will have cationic nitrogen containing groups such as quaternary ammonium or protonated amino groups, or a mixture thereof. Suitable cationic nitrogen polymers are described in the CTFA Cosmetic Ingredient Directory, 3rd edition. The cationic conditioning polymer may be present individually or as mixtures of two or more different cationic conditioning polymers.

Of particular interest as cationic polymers are cationic polysaccharides with an anhydroglucose residual group, such as a starch or cellulose Such cationic polysaccharides include, for example, quaternary cellulosic polymers. Quateranary cellulosic polymers suitable for use herein include, for example, the polymeric quaternary ammonium salts of hydroxyethyl cellulose reacted with trimethylammonium substituted epoxide, e.g., cationic polymers referred to in the industry as Polyquaternium-10; such polymers include products available from Amerchol Corp (Edison, N.J., USA), as their Polymer JR and LR series of polymers. Another type of quaternary cellulose are the polymeric quaternary ammonium salts of hydroxyethyl cellulose reacted with lauryl dimethyl ammonium-substituted epoxide, referred to in the industry (CTFA) as Polyquaternium 24; such polymers are available from Amerchol Corp (Edison, N.J., USA) under the tradename Polymer LM-200. Other suitable quaternary cellulose derivatives include quaternary nitrogen-containing cellulose ethers (e.g. as described in U.S. Pat. No. 3,962,418), and copolymers of etherified cellulose and starch (e.g. as described in U.S. Pat. No. 3,958,581).

Other quaternary polysaccharide polymers of interest in the practice of this invention are, for example, quaternary guar derivatives. Suitable quaternary guar gum derivatives include, for example, guar hydroxypropyltrimonium chloride (commercially available from Rhone-Poulenc in their Jaguar series of products). Particularly preferred quaternary guar gum derivatives are JAGUAR C13S, JAGUAR C14, JAGUAR C15, JAGUAR C17 and JAGUAR C16 Jaguar CHT and JAGUAR C162.

In at least one embodiment of interest, the quaternary polysaccharide comprises a polymer of the formula: 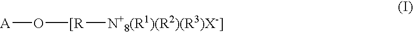
wherein A is an anhydroglucose residual group such as a starch or cellulose anhydroglucose residue, R is an alkylene, oxyalkylene, polyoxyalkylene, or hydroxyalkylene group, or combinations thereof, wherein R¹, R², and R³ independently represent alkyl, aryl, alkaryl, aryalkyl, alkoxyalkyl or alkoxyaryl groups, each group containing up to about 18 carbon atoms. The total number of carbon atoms for each quaternary moiety (i.e., the sum of carbon atoms in R¹, R² and R³) is preferably about 20 or less, and X is an anionic counterion, preferably chloride or bromide.

Other suitable cationic conditioning polymers include, for example, copolymers of 1-vinyl-2-pyrrolidine and 1-vinyl-3-methyl-imidazolium salt (e.g., polymers having CTFA name Polyquaternium-16); copolymers of 1-vinyl-2-pyrrolidine and dimethylaminoethyl methacrylate, (e.g., polymers having the CTFA name Polyquaternium-11); cationic diallyl quaternary ammonium-containing polymers (e.g., polymers having the CTFA name Polyquaternium-6) and quaternary ethaminium polymers (e.g., polymers having the CTFA Polyquaternium-32 and Polyquaternium-37). Other suitable cationic conditioning polymers are mineral acid salts of amino-alkyl esters of homo- and co-polymers of unsaturated carboxylic acids as described in U.S. Pat. No. 4,009,256, as well cationic polyacrylamides as described in WO95/22311.

In at least one embodiment of particular interest, the cationic conditioning polymer comprises a cationic cellulosic polymer, preferably Polyquaternium-10.

The cationic conditioning polymer will generally be present in the compositions of the invention at levels of 0.01 to 5 wt. %. The level of preference depends upon the type of composition into which it is incorporated.

For example, in shampoo compositions the use of cationic conditioning polymer at a level of 0.03 to 2.0 weight %, more particularly from 0.05 to 1.0 weight %, is of particular interest. In one preferred embodiment, the shampoo composition comprises from 0.08 to 0.2 weight % of cationic conditioning polymer.

In conditioner compositions, particularly leave-on conditioner compositions, the use of cationic conditioning polymer at a level of from 0.5 to 5 weight %, more particularly from 0.8 to 4 weight %, is of particular interest. In one preferred embodiment, the conditioner composition comprises from 1-3 wt. % of cationic conditioning polymer.

In the case of styling aids, the level of preference of the cationic conditioning polymer will depend upon the form of the composition; for example, 1 to 5 weight % in the case of styling creams, compared to 0.3 to 5 weight % in the case of styling gels.

The dicarboxylic acid suitable for use in the practice of this invention comprises one or more dicarboxylic acids of the formula:
HOOC-A-COOH
wherein A is an alkylene group of the formula: —(CH₂)ₙ— where n is an integer from 2 to 8, preferably from 2 to 4, the alkylene group A being optionally substituted with a group of the formula —SO₃H. Of particular use in the practice of this invention are dicarboxylic acids wherein the alkylene group A is substituted with a group of the formula —SO₃H. In at least one preferred embodiment of this invention the dicarboxylic acid comprises sulfosuccinic acid.

The dicarboxylic acid will generally be present in the compositions of the invention at a level of from 0.5 to 5 weight percent and, at least one embodiment of interest, at a level of from 1 to 4 wt. %.

The level of preference depends upon the type of compositions into which the dicarboxylic acid is incorporated.

For example, in shampoo compositions the use of dicarboxylic acid at a level of 0.5 to 5 wt. %, more particularly, 0.8 to 3 wt. % is of particular interest. In one preferred embodiment, the shampoo composition comprises from 1 to 2.5 wt. % of the dicarboxylic acid. In conditioner compositions, particularly leave-on conditioner compositions, the use of dicarboxylic acid at a level of 0.5 to 5 wt. %, more particularly from 0.5 to 3 wt. %, is of particular interest. In one preferred embodiment, the conditioner composition comprises from 0.5 to 2 wt. % of the dicarboxylic acid component. In the case of styling aids, the level of preference of the dicarboxylic acid component the depends upon the form of the composition; for example, 0.5 to 3.0 wt. % in the case of styling creams, compared to 0.5 to 5.0 wt. % in the case of styling gels.

Optionally, some or all of the dicarboxylic acid may be in the form of a water-soluble salt thereof, including for example alkali, ammonium, and/or alkanol ammonium salts, with sodium salts being of particular interest.

Anionic Cleansing Surfactant

Shampoo compositions according to the invention will typically comprise one or more anionic cleansing surfactants, which are cosmetically acceptable and suitable for topical application to the hair.

Examples of suitable anionic cleansing surfactants are the alkyl sulfates, alkyl ether sulfates, alkaryl sulfonates, alkanoyl isethionates, alkyl succinates, alkyl sulfosuccinates, N-alkyl sarcosinates, alkyl phosphates, alkyl ether phosphates, alkyl ether carboxylates, and alpha-olefin sulfonates, especially their sodium, magnesium, ammonium and mono-, di- and triethanolamine salts. The alkyl and acyl groups generally contain from 8 to 18 carbon atoms and may be unsaturated. The alkyl ether sulfates, alkyl ether phosphates and alkyl ether carboxylates may contain from 1 to 10 ethylene oxide or propylene oxide units per molecule.

Typical anionic cleansing surfactants for use in shampoo compositions of the invention include sodium oleyl sulfosuccinate, ammonium lauryl sulfosuccinate, ammonium lauryl sulfate, sodium cocoyl isethionate, sodium lauryl isethionate and sodium N-lauryl sarcosinate. The most preferred anionic surfactants are sodium lauryl sulfate, sodium lauryl ether sulphate(n)EO, (where n ranges from 1 to 3), ammonium lauryl sulfate and ammonium lauryl ether sulfate(n)EO, (where n ranges from 1 to 3).

The total amount of anionic cleansing surfactant in shampoo compositions of the invention is generally from 5 to 30, preferably from 6 to 20, more preferably from 8 to 16 wt % of the total composition.

Co-Surfactant

The shampoo composition can optionally include co-surfactants, preferably an amphoteric or zwitterionic surfactant, which can be included in an amount ranging from 0 to about 8, preferably from 1 to 4 wt %.

Examples of amphoteric and zwitterionic surfactants include, alkyl betaines, alkyl amidopropyl betaines, alkyl sulfobetaines (sultaines), alkyl glycinates, alkyl carboxyglycinates, alkyl amphopropionates, alkylamphoglycinates, alkyl amidopropyl hydroxysultaines, acyl taurates and acyl glutamates, wherein the alkyl and acyl groups have from 8 to 19 carbon atoms. Typical amphoteric and zwitterionic surfactants for use in shampoos of the invention include lauryl amine oxide, cocodimethyl sulfopropyl is betaine and preferably lauryl betaine, cocamidopropyl betaine and sodium cocamphopropionate.

Another preferred co-surfactant is a nonionic surfactant, which can be included in an amount ranging from 0 to 8 wt %, preferably from 2 to 5 wt % of the total Composition.

Representative nonionic surfactants that can be included in shampoo compositions of the invention include condensation products of aliphatic (C₈-C₁₈) primary or secondary linear or branched chain alcohols or phenols with alkylene oxides, usually ethylene oxide and generally having from 6 to 30 ethylene oxide groups.

Further nonionic surfactants, which can be included in shampoo compositions of the invention, are the alkyl polyglycosides (APGs). Typically, the APG is one which comprises an alkyl group connected (optionally via a bridging group) to a block of one or more glycosyl groups. Preferred APGs are defined by the following formula:
RO—(G)ₙ
wherein R is a branched or straight chain C₅ to C₂₀alkyl or alkenyl group, G is a saccharide group and n is from 1 to 10.

Other sugar-derived nonionic surfactants which can be included in shampoo compositions of the invention include the C₁₀-C₁₈ N-alkyl (C₁-C₆) polyhydroxy fatty acid amides, such as the C₁₂-C₁₈ N-methyl glucamides, as described for example in WO 92 06154 and U.S. Pat. No. 5,194,639, and the N-alkoxy polyhydroxy fatty acid amides, such as C₁₀-C₁₈ N-(3-methoxypropyl) glucamide.

The total amount of surfactant (including any co-surfactant, and/or any emulsifier) in shampoo compositions of the invention is generally from 5 to 50, preferably from 5 to 30, more preferably from 10 to 25 wt %.

The volumizing benefits of the shampoo compositions of this invention may be enhanced by providing the composition with surfactant that has a swelling effect on the keratin protein. Without wishing to be bound to theory, it is believed that swelling of the hair fiber may promote absorption of the dicarboxylic acid which in turn, may have a stiffening effect on the hair fiber. Zein solubility provides a measure of a composition's ability to swell keratin protein. Throughout the specification and claims “Zein Solubility” refers to Zein solubility as measured by the Zein Solubility Methodology described below. In a preferred embodiment, the compositions of this invention, particularly the shampoo compositions of this invention, have a Zein Solubility of at least 2.0, preferably at least 2.4.

When used in conjunction with a post-shampoo conditioner, the body/volume properties of hair treated with a shampoo composition according to this invention may be impacted by composition of the post-shampoo conditioner with which it is used. It has been found that conditioner composition comprising:
A) from 1 to 10 wt. %, of fatty alcohol, and
B) from 0.1 to 10 wt. % of cationic conditioning surfactant comprising a mixture of mono- and dialkyl quats, wherein the ratio of monoalkyl quat to dialkyl quat in the conditioning composition is from 1:1 to 5:1, preferably 1:1 to 3:1, on an active basis, and
C) water,
   are particularly well suited for use with the shampoo compositions of this invention. Accordingly, in an embodiment of particular interest there is provided a system for treating and conditioning hair which system comprises, as individually packaged components:
A) a shampoo composition comprising:
   a) from 0.01 to 5 wt. % of cationic conditioning polymer; and
   b) from 0.5 to 5 wt. % of dicarboxylic acid of the formula:
      HOOC-A-COOH
   wherein A is an alkylene group of the formula: —(CH₂)ₙ—, where n is an integer from 2 to 8, the alkylene group A being optionally substituted with a group of the formula —SO₃H, and wherein from 0 to 100% of the dicarboxylic acid is a water-soluble salt thereof; and
B) a conditioner composition comprising:
   a) from 1 to 10 wt. % of fatty alcohol, and
   b) from 0.1 to 10 wt. % of cationic conditioning surfactant comprising a mixture of mono- and dialkyl quats, wherein the ratio of monoalkyl quat to dialkyl quat in the conditioning composition is from 1.1 to 5:1, and
   c) water.
      Mono- and dialkylquats suitable for use in the conditioning compositions of the subject systems are described below under “conditioning surfactants”.
      Conditioning Surfactant

Depending upon the particular form thereof, the compositions of this invention may desirably further comprise one or more conditioning surfactants, which are cosmetically acceptable and suitable for topical application to the hair. The use of conditioning surfactants is of particular interest when the composition is formulated as a hair conditioner.

Suitable conditioning surfactants are selected from cationic surfactants, used singly or in a mixture.

Cationic surfactants useful in compositions of the invention contain amino or quaternary ammonium hydrophilic moieties, which are positively charged when, dissolved in the aqueous composition of the present invention.

Examples of suitable cationic surfactants are those corresponding to the general formula:
[N(R₁)(R₂)(R₃)(R₄)]⁺(X)⁻ (II)
in which R₁, R₂, R₃, and R₄ are independently selected from (a) an aliphatic group of from 1 to 22 carbon atoms, or (b) an aromatic, alkoxy, polyoxyalkylene, alkylamido, hydroxyalkyl, aryl or alkylaryl group having up to 22 carbon atoms; and X is a salt-forming anion such as those selected from halide, (e.g. chloride, bromide), acetate, citrate, lactate, glycolate, phosphate nitrate, sulphate, and alkylsulphate radicals.

The aliphatic groups can contain, in addition to carbon and hydrogen atoms, ether linkages, and other groups such as amino groups. The longer chain aliphatic groups, e.g., those of about 12 carbons, or higher, can be saturated or unsaturated. Included among the conditioning surfactants are mono- and dialkyl quats. Monoalkyl quats suitable for use herein include Formula (II) conditioning surfactants wherein R₁, R₂, and R₃, which may be the same or different, are C1-C4 hydrocarbyl groups and R₄ is a C8 or greater hydrocarbyl group (preferably C16 to C22 alkyl). The monoalkyl quats may, but need not be in the form of mixtures of monoalkyl quats.

Also included among the Formula II conditioning surfactants are dialkyl quats in which R₁ and R₂, which may be the same or different are C1-C4 hydrocarbyl groups and R₃, and R₄, which may be the same or different are C8 or greater alkyl groups (preferably C14 to C22 alkyl). The dialkyl quats may, but need not be in the form of mixtures of dialkyl quats.

Nonlimiting examples of cationic conditioning surfactants include cetyltrimethylammonium chloride, stearyltrimethylammonium chloride, behentrimonium chloride, tallowtrimonium chloride, palmityltrimethylammonium chloride, cocotrimonium chloride, hydrogenated tallowtrimethylammonium chloride, benzyltrimethylammonium chloride, cetylpyridinium chloride, cetrimonium tosylate, dimethyldicetylammonium chloride, dimethyldistearylammonium chloride, dimethyldipalmitylammonium chloride, dimethyl(dihydrogenated tallow)ammonium chloride, tetraethylammonium chloride, PEG-2 oleylammonium chloride and salts of these where the chloride is replaced by halide (e.g., bromide), acetate, citrate, lactate, glycolate, phosphate nitrate, sulfate, or alkylsulfate. Further suitable cationic surfactants include those materials having the CTFA designations Quaternium-5, Quaternium-31 and Quaternium-18. Mixtures of any of the foregoing materials may also be suitable. A particularly useful cationic surfactant for use in hair conditioners of the invention is cetyltrimethylammonium chloride, available commercially, for example as GENAMIN CTAC, ex Hoechst Celanese.

Salts of primary, secondary, and tertiary fatty amines are also suitable cationic surfactants. The alkyl groups of such amines preferably have from 12 to 22 carbon atoms, and can be substituted or unsubstituted.

Particularly useful are amido substituted tertiary fatty amines, in particular tertiary amines having one C₁₂ to C₂₂ alkyl or alkenyl chain. Such amines, useful herein, include stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Also useful are dimethylstearamine, dimethylsoyamine, soyamine, myristylamine, tridecylamine, ethylstearylamine, N-tallowpropane diamine, ethoxylated (with 5 moles of ethylene oxide) stearylamine, dihydroxyethylstearylamine, and arachidyl behenylamine.

These amines are typically used in combination with an acid to provide the cationic species. The preferred acid useful herein includes L-glutamic acid, lactic acid, hydrochloric acid, malic acid, succinic acid, acetic acid, fumaric acid, tartaric acid, citric acid, L-glutamic hydrochloride, and mixtures thereof; more preferably L-glutamic acid, lactic acid, citric acid. Cationic amine surfactants included among those useful in the present invention are disclosed in U.S. Pat. No. 4,275,055 to Nachtigal, et al., issued Jun. 23, 1981.

The molar ratio of protonatable amines to H+ from the acid is preferably from about 1:0.3 to 1:1.2, and more preferably from about 1:0.5 to about 1:1.1.

In the conditioners of the invention, the level of cationic surfactant is preferably present in an amount of from 0.01 to 10, more preferably 0.05 to 5, most preferably 0.1 to 2 wt % of the total composition.

Fatty Materials

The compositions of the invention preferably additionally comprise fatty materials, particularly if such compositions are formulated as rinse-off conditioners or leave-on creams. The combined use of fatty materials and cationic surfactants in conditioning compositions is believed to be especially advantageous, because this leads to the formation of a structured phase, in which the cationic surfactant is dispersed.

By “fatty material” is meant a fatty alcohol, an alkoxylated fatty alcohol, a fatty acid or a mixture thereof.

Preferably, the alkyl chain of the fatty material is fully saturated.

Representative fatty materials comprise from 8 to 22 carbon atoms, more preferably 16 to 22. Examples of suitable fatty alcohols include cetyl alcohol, stearyl alcohol and mixtures thereof. The use of these materials is also advantageous in that they contribute to the overall conditioning properties of compositions of the invention.

Alkoxylated, (e.g. ethoxylated or propoxylated) fatty alcohols having from about 12 to about 18 carbon atoms in the alkyl chain can be used in place of, or in addition to, the fatty alcohols themselves. Suitable examples include ethylene glycol cetyl ether, polyoxyethylene (2) stearyl ether, polyoxyethylene (4) cetyl ether, and mixtures thereof.

The level of fatty alcohol material in conditioners of the invention is suitably from 0.01 to 15, preferably from 0.1 to 10, and more preferably from 0.1 to 5 wt %. The weight ratio of cationic surfactant to fatty alcohol is suitably from 10:1 to 1:10, preferably from 4:1 to 1:8, optimally from 1:1 to 1:7, for example 1:3.

Suspending Agents

The hair treatment composition, especially if it is a shampoo composition, optimally further comprises a suspending agent. Suitable suspending agents are selected from polyacrylic acids, cross-linked polymers of acrylic acid, copolymers of acrylic acid with a hydrophobic monomer, copolymers of carboxylic acid-containing monomers and acrylic esters, cross-linked copolymers of acrylic acid and acrylate esters, heteropolysaccharide gums and crystalline long chain acyl derivatives. The long chain acyl derivative is desirably selected from ethylene glycol stearate, alkanolamides of fatty acids having from 16 to 22 carbon atoms and mixtures thereof. Ethylene glycol distearate and polyethylene glycol 3 distearate are preferred long chain acyl derivatives. Polyacrylic acid is available commercially as Carbopol 907. Polymers of acrylic acid cross-linked with a polyfunctional agent may also be used; they are available commercially as Carbopol 910, Carbopol 934, Carbopol 941 and Carbopol 980. An example of a suitable copolymer of a carboxylic acid containing monomer and acrylic acid esters is Carbopol 1342. All Carbopol (trademark) materials are available from Goodrich. Suitable cross-linked polymers of acrylic acid and acrylate esters are Pemulen TR1 or Pemulen TR2. A suitable heteropolysaccharide gum is xanthan gum, for example that available as Kelzan mu. When present, the suspending agents typically comprise from 0.1 to 5 wt. %, more particularly from 0.2 to 1 wt. % of the hair treatment agents.

Additional Conditioning Agents

Optionally, the compositions of the invention can include one or more additional hair conditioning agents such as are commonly present such in hair treatment compositions, provided that the additional conditioning agents, in the amounts in which they are used, do not undesirably impact the bodifying/volume properties of the subject compositions. Exemplary of additional conditioning agents, are, for example, silicone containing conditioners such as for example, polydiorganosiloxanes, in particular polydimethylsiloxanes that have the CTFA designation dimethicone, as well as polydimethyl siloxanes having hydroxyl end groups, which have the CTFA designation dimethiconol. Also suitable for use in compositions of the invention are silicone gums having a slight degree of cross-linking, as are described for example in WO 96/31188.

The viscosity of the emulsified silicone itself (not the emulsion or the final hair conditioning composition) is typically at least 10,000 cst at 25° C. the viscosity of the silicone itself is preferably at least 60,000 cst, most preferably at least 500,000 cst, ideally at least 1,000,000 cst. Preferably the viscosity does not exceed 10⁹ cst for ease of formulation.

Emulsified silicones for use in the compositions of the invention will typically have an average silicone droplet size in the composition of less than 30, preferably less than 20, more preferably less than 10 μm, ideally from 0.01 to 1 μm. Silicone emulsions having an average silicone droplet size of ≦0.15 μm are generally termed microemulsions.

Examples of suitable pre-formed emulsions include emulsions DC2-1766, DC2-1784, DC-1785 DC-1786 and microemulsions DC2-1865 and DC2-1870, all available from Dow Corning. These are all emulsions/microemulsions of dimethiconol. Cross-linked silicone gums are also available in a pre-emulsified form, which is advantageous for ease of formulation. A preferred example is the material available from Dow Corning as DC X2-1787, which is an emulsion of cross-linked dimethiconol gum. A further preferred example is the material available from Dow Corning as DC X2-1391, which is a microemulsion of cross-linked dimethiconol gum.

A further preferred class of silicones for inclusion in shampoos and conditioners of the invention are amino functional silicones. By “amino functional silicone” is meant a silicone containing at least one primary, secondary or tertiary amine group, or a quaternary ammonium group. Examples of suitable amino functional silicones include: polysiloxanes having the CTFA designation “aminodimethicone”.

Specific examples of amino functional silicones suitable for use in the invention are the aminosilicone oils DC2-8220, DC2-8166, DC2-8466, and DC2-8950-114 (all ex Dow Corning), and GE 1149-75, (ex General Electric Silicones).

When present, such additional conditioning agents are typically used at a level of from 0.5 to 5 weight %, depending upon the form of the composition into which they are incorporated.

In one preferred embodiment, the shampoo compositions are “substantially free” of these additional conditioning agents. As used herein “substantially free” means an amount of from 0 to about 0.5 percent by weight, based on the total weight of the shampoo composition.

Styling Polymers

If the product is a styling product it is preferred that a styling polymer is present. The hair styling polymer, if present, is preferably present in the compositions of the invention in an amount of from 0.001% to 10% by weight, more preferably from 0.1% to 10% by weight, such as from 1% to 8% by weight.

Hair styling polymers are well known. Suitable hair styling polymers include commercially available polymers that contain moieties that render the polymers cationic, anionic, amphoteric or nonionic in nature. Suitable hair styling polymers include, for example, block and graft copolymers. The polymers may be synthetic or naturally derived.

Other Optional Ingredients

One or more additional ingredients as are conventionally incorporated into hair treatment compositions may be included in compositions of the invention. Such additional ingredients include, for example, fragrances, perfumes, dyes, coloring agents, buffering or pH adjusting agents, deposition aids, chelating agents, viscosity modifiers, thickening agents, suspending agents, opacifiers, pearlizers, antidandruff agents, sunscreens, antioxidants, preservatives, antibacterial agents, proteins, amino acids, moisturizing agents, herb or other plant extracts and other natural ingredients.

The hair treatment of this invention may be prepared by conventional techniques known to those skilled in the art.

The invention is further illustrated by way of the following non-limiting examples. Unless otherwise indicated, all parts and percentages are by weight.

### EXAMPLES

### Example 1

Shampoo compositions were formulated by preparing emulsions having the formulations described in Table 1.

The Zein Solubility of the shampoo compositions was measured by means of the following methodology. Zein Solubility values of the shampoo compositions is reported in Table 1.

Zein Solubility Methodology

Zein is a protein (blends of amino acid derived from maize) which swells and denatures in response to surfactants in a similar way to skin keratin proteins. In general, compositions (e.g., shampoos) that are more swelling to hair fiber have higher Zein Solubility values than compositions that are less swelling to the hair fiber.

Apparatus

Analytical balance, 100 ml beakers, stir bars, medium stir plate, 10 ml syringe, 20 ml scintillation vials, conventional oven, set at 75° C.

Procedure

1. Weigh 6.25 g of shampoo into a 100-ml beaker and dilute it to 50 g with deionized water.
2. Mix the solution on a stir plate @ 300 rpm (set dial at 4 on stirring plate) until the solution looks uniform or the entire sample is dissolved.
3. Record the pH of the solution.
4. Withdraw 6 ml of solution using a syringe.
5. Filter solution through a 0.45-micron syringe filter onto a scintillation vial.
6. Cap the vial and label it as blank. A blank is needed to correct for any soluble material.
7. Add 2 g of Zein to the remaining solution and equilibrate for 1 hour at constant stirring speed (300 rpm). After 10 minutes of stirring, if all or most of the Zein dissolved, add an additional 1 g of Zein. Keep adding more Zein in 1 g increments every 5-10 minutes until there is undissolved Zein floating in the solution.
8. After 1 hour of constant stirring, allow solution to settle for 5 minutes.
9. Withdraw 6 ml of the supernatant solution using a syringe and filter it through a 0.45 micron syringe filter onto a scintillation vial.
10. Cap the vial and label it as sample.
11. Perform nonvolatile analysis on both samples using a conventional oven set at 75° C. Allow samples to dry overnight.
12. Calculate the percent Zein dissolved.
   Calculation
   % Zein solubilized=% nonvolatile of sample−% nonvolatile of blank

TABLE 1
SHAMPOO COMPOSITONS

| Component (wt. % on 100% active | | | | | | |
|---|---|---|---|---|---|---|
| basis) | A | B | C | D | E | F |
| Sodium laureth sulfate (1 EO) | 14.0 | 6.0 | 14.0 | 14.0 | 6.0 | 6.0 |
| Cocamidopropyl betaine | 1.6 | 3.0 | 1.6 | 1.6 | 3.0 | 3.0 |
| Disodium laureth sulfosuccinate | — | 4.0 | — | — | 4.0 | 4.0 |
| Polyquaternium-10 | 0.1 | 0.1 | 0.1 | — | — | — |
| Sulfosuccinic acid* | — | 1.0 | 1.0 | — | 1.0 | 1.0 |
| Modified silica | — | — | — | 0.6 | 0.6 | — |
| Dimethiconol | — | — | — | 2.0 | 2.0 | — |
| Guar hydroxypropyltrimonium chloride | — | — | — | 0.1 | 0.1 | 0.1 |
| Cross-linked silicone | — | — | — | — | — | 0.6 |
| Opacifier (ethylene glycol distearate | — | 1.0 (EGDS) | — | 0.2 | 1.0 (EGDS) | 1.0 (EGDS) |
| (EGDS) or mica/TiO2) | | | | (mica/TiO2) | | |
| Thickener | — | — | — | 0.6 | 0.6 | 0.4 |
| Fragrance | 0.5 | 0.5 | 0.5 | 0.5 | 0.5 | 0.5 |
| Preservative | 0.3 | 0.3 | 0.3 | 0.3 | 0.3 | 0.3 |
| pH Adjuster | To 5.5 | To 5.5 | To 5.5 | To 5.5 | To 5.5 | To 5.5 |
| Viscosity Adjuster | To approx. | To approx. | To approx. | To approx. | To approx. | To approx. |
| | 7000 cps | 7000 cps | 7000 cps | 7000 cps | 7000 cps | 7000 cps |
| Deionized water | Q.S. | Q.S. | Q.S. | Q.S. | Q.S. | Q.S. |
| Zein Solubility | 3.0 | <2.0 | 3.2 | | | |

*In the case of compositions containing disodium laureth sulfosuccinate, the amount of sulfosuccinic acid reported includes sulfosuccinic acid present as an impurity in the disodium laureth sulfosuccinate.

Hair conditioner having the formulation described in Table 2 were prepared

TABLE 2
Conditioner Composition (G Conditioner)

| Description | % wt/wt (as supplied) |
|---|---|
| Deionized water | 81.31 |
| Hydroxyethylcellulose | 0.20 |
| Cetrimonium chloride, 30% active | 5.60 |
| Potassium Chloride | 0.10 |
| Cetearyl Alcohol | 6.00 |
| Quaternium —18, 75% active | 1.00 |
| Disodium EDTA | 0.10 |
| Dimethiconol, 60% active | 2.50 |
| Cyclopentasiloxane | 1.70 |
| Glycerin | 1.00 |
| DMDM Hydantoin preservative | 0.14 |
| Fragrance | 0.35 |

Shampoo compositions A through F were evaluated by a study panel made up of 183 female panelists, through screening, identified as body/volume seekers who shampooed and conditioned their hair 3+times per week. Over a period of four weeks, each panelist received four pairs of shampoo and conditioner for evaluation (1 pair per week). Samples to were randomized so as to promote balance with respect to the shampoo samples being seen equally often. The panelists were instructed to use the pair of shampoo and conditioner received in a given week in place of their current shampoo and conditioner at least 4 times during that week (no other shampoos and conditioners were to be used during this period). Unknown to the panelists, the conditioner composition remained constant (G conditioner). The panelists were asked to rate the properties of the shampooed, conditioned hair on a 9-point scale. Test results are reported in Table 3.

TABLE 3

| | | | | | |
|---|---|---|---|---|---|
| A Shampoo/ | B Shampoo/ | C Shampoo/G | D Shampoo/ | E Shampoo/ | F Shampoo/ |
| G Conditioner | G Conditioner | Conditioner | G Conditioner | G Conditioner | G Conditioner |

Intensity for Shampoo/Conditioner System (9-point scale)

| | | | | | | | | | | | | |
|---|---|---|---|---|---|---|---|---|---|---|---|---|
| Ease of detangling of wet hair | 7.0 | abAB | 6.5 | cB | 7.2 | aA | 6.8 | bcAB | 6.8 | bcAB | 6.5 | cB |
| Intensity of fragrance on dry hair | 4.2 | b | 4.7 | a | 4.3 | ab | 4.4 | ab | 4.6 | a | 4.6 | a |
| Ease of combing/brushing of dry | 7.0 | | 6.8 | | 7.1 | | 7.0 | | 7.0 | | 6.8 | |
| hair | | | | | | | | | | | | |
| Coated feel of dry hair | 5.0 | | 5.2 | | 5.1 | | 5.0 | | 5.2 | | 5.1 | |
| Clean feel of dry hair | 6.6 | | 6.5 | | 6.8 | | 6.5 | | 6.6 | | 6.7 | |
| Amount of conditioning of dry hair | 6.3 | AB | 6.3 | AB | 6.6 | A | 6.1 | B | 6.3 | AB | 6.0 | B |
| Ease of styling | 5.9 | | 6.1 | | 6.1 | | 6.0 | | 6.0 | | 6.3 | |
| Manageability | 5.7 | b | 6.1 | ab | 6.1 | ab | 5.8 | ab | 5.9 | ab | 6.2 | a |
| Amount of volume/fullness | 5.1 | | 5.6 | | 5.6 | | 5.3 | | 5.2 | | 5.6 | |
| How thick it left hair | 4.6 | bB | 5.2 | aA | 5.3 | aA | 5.0 | abAB | 4.9 | abAB | 5.2 | aAB |
| Amount of bounce | 4.9 | bB | 5.4 | AAB | 5.5 | aA | 5.1 | abAB | 5.1 | abAB | 5.3 | abAB |
| Amount of body | 5.0 | cB | 5.5 | AbAB | 5.7 | aA | 5.3 | abcAB | 5.1 | bcAB | 5.5 | abAB |
| Amount of dryness | 3.5 | aAB | 3.5 | AAB | 3.0 | bB | 3.5 | aAB | 3.7 | aA | 3.6 | aA |
| Amount of build-up left on hair | 3.4 | | 3.6 | | 3.7 | | 3.6 | | 3.9 | | 3.7 | |
| Amount of greasiness | 2.9 | | 2.9 | | 3.1 | | 3.1 | | 3.2 | | 3.0 | |
| How flat/limp it left hair | 4.6 | | 4.3 | | 4.2 | | 4.3 | | 4.5 | | 4.4 | |
| Amount of softness | 6.6 | b | 7.0 | a | 6.9 | ab | 6.5 | b | 6.7 | ab | 6.6 | b |
| How smooth it left hair | 6.5 | ab | 6.7 | ab | 6.9 | a | 6.5 | b | 6.6 | ab | 6.5 | ab |
| Amount of shine | 6.0 | AB | 6.2 | A | 6.3 | A | 5.7 | B | 6.0 | AB | 6.2 | A |
| Amount of flyaways | 4.1 | | 3.9 | | 3.9 | | 4.0 | | 3.9 | | 4.2 | |
| Amount of static | 3.2 | | 3.2 | | 3.3 | | 3.2 | | 3.2 | | 3.4 | |

*Row means sharing the same letter, or no letter, are not significantly different at 90% (lower case letters) or 95% (upper case letters) confidence levels.

Conditioner Composition

The following composition is but one example of a leave-on conditioner as described by this invention.

TABLE 4

| Component | Weight % |
|---|---|
| Deionized water | 50 |
| Adipic acid | 0.73 |
| CTAC, 30% (active) | 1.7 |
| Cetearyl alcohol | 2 |
| Polysurf 67 | 0.06 |
| Deionized water | Qs |
| DC 1787 | 0.66 |
| Polyquat-16 | 2.25 |
| Glydant plus liquid | 0.4 |
| Fragrance | 0.3 |
| Deionized water | 2 |
| EDTA disodium | 0.1 |
| NaOH | To pH 4 |

What is claimed is:

1. A hair treatment composition comprising:
a) from 0.01 to 5 weight percent of cationic conditioning polymer; and
b) from 0.5 to 5 weight percent of dicarboxylic acid of the formula:
HOOC-A-COOH
wherein A is an alkylene group of the formula: —(CH₂)ₙ— where n is an integer from 2 to 8, the alkylene group A being optionally substituted with a group of the formula —SO₃H,
and wherein from 0 to 100% of the dicarboxylic acid is a water-soluble salt thereof.

2. A hair treatment composition as described in claim 1 wherein the cationic conditioning polymer comprises at least one cationic polysaccharide.

3. A hair treatment composition as described in claim 2 wherein the cationic conditioning polymer comprises at least one cationic cellulosic polymer.

4. A hair treatment composition as described in claim 1 wherein the cationic conditioning polymer comprises Polyquaternium-10.

5. A hair treatment composition as described in claim 1 wherein the alkylene group A of the dicarboxylic acid is substituted with a group of the formula —SO₃H.

6. A hair treatment composition as described in claim 1 where n is an integer from 2 to 4.

7. A hair treatment composition as described in claim 1 wherein the dicarboxylic acid comprises sulfosuccinic acid.

8. A hair treatment composition as described in claim 1 which is a shampoo.

9. A hair treatment composition as described in claim 8 wherein the dicarboxylic acid is present in the composition an amount of from 0.8 to 3 wt. %, and wherein from 0 to 100% of the dicarboxylic acid is a water-soluble salt thereof.

10. A hair treatment composition as described in claim 8 wherein the hair treatment composition has a Zein Solubility value of at least 2.0.

11. A shampoo composition comprising:
(a) from 0.03 to 2.0 weight percent of at least one cationic conditioning polymer;
(b) from 0.8 to 3 weight percent of sulfosuccinic acid, and
(c) anionic surfactant,
and wherein from 0 to 100% of the sulfosuccinic acid is a water-soluble salt thereof.

12. A shampoo composition as described in claim 11 having a Zein Solubility value of at least 2.4.

13. A shampoo composition as described in claim 11 wherein the cationic conditioning polymer comprises at least one cationic polysaccharide.

14. A hair treatment composition as described in claim 1 wherein the dicarboxylic acid is present in an amount of from 0.8 to 3 wt. %.

15. A hair treatment composition as described in claim 1 wherein the dicarboxylic acid is present in an amount of 1 to 2.5 weight %.

16. A system for treating and conditioning hair which system comprises, as individually packaged components:
A) a shampoo composition comprising:
a) from 0.01 to 5 wt. % of cationic conditioning polymer; and
b) from 0.5 to 5 wt. % of dicarboxylic acid of the formula:
HOOC-A-COOH
wherein A is an alkylene group of the formula: —(CH₂)ₙ—, where n is an integer from 2 to 8, the alkylene group A being optionally substituted with a group of the formula —SO₃H, and wherein from 0 to 100% of the dicarboxylic acid is a water-soluble salt thereof; and
B) a conditioner composition comprising:
a) from 1 to 10 wt. %, fatty alcohol, and
b) from 0.1 to 10 wt. % of cationic conditioning surfactant comprising a mixture of mono- and dialkyl quats, wherein the ratio of monoalkyl quat to dialkyl quat in the conditioning composition is from 1:1 to 5:1, and
c) water.

17. A hair treatment composition as described in claim 1 wherein the cationic conditioning polymer consists essentially of at least one cationic polysaccharide.

18. A hair treatment composition as described in claim 1 wherein the dicarboxylic acid consists essentially of sulfosuccinic acid, wherein from 0 to 100% of the sulfosuccinic acid is a water-soluble salt thereof.
